(19) United States
(12) Patent Application Publication
(10) Pub. No.: US 2021/0147366 A1
(43) Pub. Date: May 20, 2021

(54) PROCESS FOR PREPARATION OF 2,2-DIMETHYLPIPERAZINE

(71) Applicant: H. Lundbeck A/S, Valby (DK)

(72) Inventors: Carla De Faveri, Farra Di Soligo TV (IT); Florian Anton Martin Huber, Dolo VE (IT); Mariano Stivanello, Schio (VI) (IT)

(21) Appl. No.: 17/045,045

(22) PCT Filed: Apr. 5, 2019
(86) PCT No.: PCT/EP2019/058600

§ 371 Date: Oct. 2, 2020

(30) Foreign Application Priority Data
Apr. 6, 2018 (DK) PA201800148

(51) Int. Cl. C07D 241/04; B01J 23/44

(52) U.S. Cl. CPC C07D 241/04; B01J 23/44

(57) ABSTRACT

This invention relates to a novel chemical process for the synthesis of 2,2-dimethylpiperazine and the further transformation of 2,2-dimethylpiperazine into ferf-butyl-3,3-dimethylpiperazine-1-carboxylate-hemi-DL-tartrate.

This invention relates to a novel chemical process for the synthesis of 2,2-dimethylpiperazine and the further transformation of 2,2-dimethylpiperazine into tert-butyl-3,3-dimethylpiperazine-1-carboxylate or a salt thereof, such as tert-butyl-3,3-dimethylpiperazine-1-carboxylate-hemi-DL-tartrate.

### BACKGROUND

2,2-dimethylpiperazine finds widespread use as an intermediate in the synthesis of a large variety of more complex compounds.

2,2-dimethylpiperazine has the following chemical formula (I):

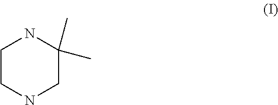

and CAS RN 84477-72-5, whereas tert-butyl-3,3-dimethylpiperazine-1-carboxylate has the chemical formula (II):

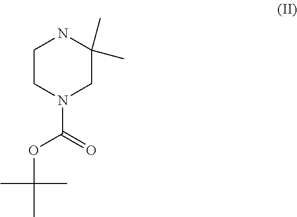

and CAS RN 259808-67-8

The hemi-tartrate of tert-butyl-3,3-dimethylpiperazine-1-carboxylate has the chemical formula (III):

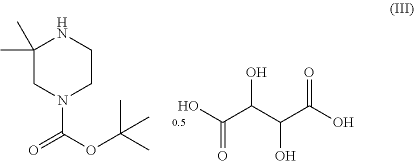

WO 2005/016900 discloses the synthesis of 3,3-dimethyl-piperazin-2-one via reaction of ethyl 2-bromo-2-methylpropanoate (also termed ethyl 2-bromoisobutyrate) with ethylenediamine in a toluene suspension in the presence of potassium carbonate. The obtained 3,3-dimethyl-piperazin-2-one is filtered and dried and finally suspended in tetrahydrofuran (THF) and subsequently reduced to 2,2-dimethylpiperazine in the presence of lithium aluminium hydroxide (LAH). The overall yield in this process is satisfactory, but the method suffers from a number of drawbacks:
ethyl 2-bromo-2-methylpropanoate is classified class 1B mutagen (H340)
work-up requires the filtration of a large amount of inorganic salts (KBr and KHCO3).
WO 2005/016900 discloses a semi-production scale, but up-scaling gives rise to a number of problems including isolation problems due to the presence of oily oligomeric by-products and as a consequence the use of large amounts of solvent (THF or toluene).
use of large quantities of lithium aluminum hydride (LAH)-THF solution (economic and safety issues linked to the use of LAH)

The above drawbacks leads to an expensive process with low productivity.

Bøgesø discloses a method of synthesising 2,2-dimethylpiperazine (Bøgesø, K. P. et al, J. Med. Chem. 1995, 38, 4380-4392) wherein isobutyraldehyde is brominated with bromine in dioxane to give 2-bromoisobutyraldehyde. Bromov aldehyde is reacted with ethylenediamine in toluene first at 5° C.-10° C. then at reflux temperature yielding 6,6-dimethyl-1,2,5,6-tetrahydropyrazine. Hydrogenation of the afforded hydropyrazine over Pd/C at low pressure provides 2,2-dimethylpiperazine. The method has been applied in WO 2008/134035 and WO 2007/127175. The method is outlined in Scheme 1 below:

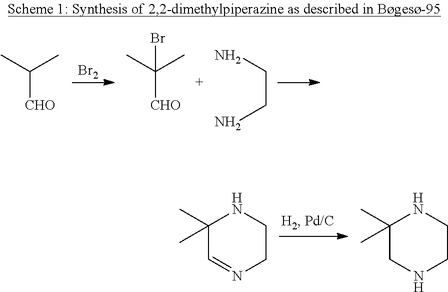

This method also suffers from a number of shortcomings, among others the use of bromine, which is both toxic and difficult to handle, a number of distillation steps are required in this method and time consuming reextractions in toluene are necessary to obtain an acceptable yield.

Thus, there is an apparent need to develop improved processes for the preparation of 2,2-dimethylpiperazine, which are cost-effective, industrially amenable, with acceptable yield and which may overcome the drawbacks of the above disclosed processes.

Rodig et. al. discloses a four step synthesis of the cyclic trimer of CO2, said synthesis includes the chlorination of isobutyraldehyde and the subsequent cyclotrimerization of 2-chloro-2-methylpropanal to form 2,4,6-tris(2-chloropropan)-2-yl-1,3,5-trioxane.

A process for obtaining tert-butyl-3,3-dimethylpiperazine-1-carboxylate hemi-DL-tartrate with 2,2-dimethylpiperazine as a starting material is disclosed in WO 2014/096151, WO 2008/019372, WO 2013/130660, WO 2007/127175 and WO 2012/124696.

### SUMMARY OF THE INVENTION

The present invention provides a novel industrially applicable process for the preparation of of 2,2-dimethylpiperazine. 2,2-Dimethylpiperazine may be purified by distillation or transformed into a suitable salt. Further, the obtained 2,2-dimethylpiperazine may be transformed into a salt of tert-butyl-3,3-dimethylpiperazine-1-carboxylate, such as tert-butyl-3,3-dimethylpiperazine-1-carboxylate hemi-DL-tartrate.

In one embodiment the invention can be summarized in steps 1-7 as outlined below. It is understood that the summary below is not limiting but illustrates a particular embodiment of the invention:
Step 1: Isobutyraldehyde is reacted with a chlorinating agent, such as sulfuryl chloride in the absence of a solvent. Gaseous by-products (sulfurdioxide and HCl) are liberated during the reaction. At the end water may be added to quench the small excess of chlorinating agent used.
Optionally the intermediate 2-chloro-2-methylpropanal is diluted with organic solvent, e.g. toluene, and treated with catalytic amounts of acidic catalyst (e.g. sulfuric acid or methanesulfonic acid) at hot to transform polymeric/trimeric forms of 2-chloro-2-methylpropanal back into 2-chloro-2-methylpropanal monomer.
Step 2:The intermediate 2-chloro-2-methylpropanal is reacted with ethlyenediamine (ETAM) in an organic solvent, e.g. THF, at hot. The imine 6,6-dimethyl-1,2,3,6-tetrahydropyrazine is obtained as a solution and used as such in the third step. Optionally re-extraction with organic solvent such as THF may be applied to recover 5-10% yield.
Step 3: The intermediate 6,6-dimethyl-1,2,3,6-tetrahydropyrazine is diluted with methanol and subjected to catalytic hydrogenation, e.g. by use of Pd/C catalyst. After removal of hydrogen and catalyst the mixture is concentrated to yield 2,2-dimethylpiperazine (containing ETAM and traces of solvent(s)). When adding more methanol (+1 Volume), the amount of palladium can be reduced by ⅓.
Step 4: Optionally crude 2,2-dimethylpiperazine obtained in Step 3 is distilled to provide pure 2,2-dimethylpiperazine.

Step 5: Optionally crude 2,2-dimethylpiperazine obtained in Step 3 is mixed with a suitable acid to provide a 2,2-dimethylpiperazine salt.

Step 6: Optionally distilled 2,2-dimethylpiperazine obtained in step 4 is mixed with a suitable acid to provide a 2,2-dimethylpiperazine salt.

Step 7: In a further step 2,2-dimethylpiperazine may be transformed into tert-butyl-3,3-dimethylpiperazine-1-carboxylate hemi-DL-tartrate by reaction with di-tert-butyl dicarbonate and addition of DL-tartaric acid.

The process as described above is recapped in Scheme 2, below:

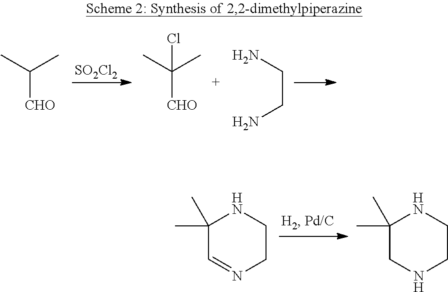

In a modified process the solid trimer 2,4,6-tris(2-chloropropan-2-yl)-1,3,5-trioxane is used as a starting material instead of isobutyraldehyde. 2,4,6-tris(2-chloropropan-2-yl)-1,3,5-trioxane has the following chemical formula (IV)

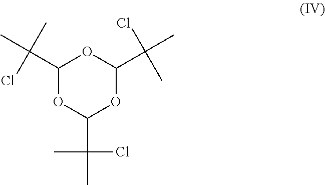 [SMILES: CC(C)(Cl)C1OC(C(C)(C)Cl)OC(C(C)(C)Cl)O1]

and CAS RN 7471-98-9. The solid trimer can be obtained upon treatment of 2-chloro-2-methylpropanal with an acid catalyst at cold. 2,4,6-tris(2-chloropropan2-yl)-1,3,5-trioxane is depolymerised by heating to a temperature above 90° C. in toluene in the presence of an acid catalyst (such as sulfuric acid, methanesulfonic acid, p-toluenesulfonic acid, or Montmorillonite K10 (CAS Number: 1318-93-0)) to obtain 2-chloro-2-methylpropanal. Step 2, Step 3 and optionally Step 4, 5 or 6 as described above are then applied to convert 2-chloro-2-methylpropanal into 2,2-dimethylpiperazine or a salt thereof.

Definitions

In the present context, “2,2-dimethylpiperazine or a salt thereof” indicates 2,2-dimethylpiperazine on its free base form or an acid addition salt which may for example be selected from tartrate, fumarate, succinate, hydrochloride, oxalate, hydrobromide, hydroiodide, sulfate, p-toluenesulfonate (tosylate) or maleate. Within the scope of the current invention is all possible stoichiometric and non stoichiometric forms of the salts of 2,2-dimethylpiperazine.

Where salts comprise one or more chiral centers, reference to the salt includes, unless otherwise specified, racemic form as well as enantiomers in any degree of purity. E.g. the term “tartrate” refers to the racemic mixture DL-tartrate as well as the enantiomers L-(+)-tartrate and D-(−)-tartrate and mixtures thereof in any ratio.

The “molar yield” is calculated on basis of the relevant starting material, e.g. ethyl 2-bromo-2-methylpropanoate or isobutyraldehyde, and its conversion to the final product, e.g. 2,2-dimethylpiperazine.

The term “Room temperature” or “Ambient temperature” means a temperature where the compound(s) and/or reaction is neither cooled nor heated; in general room temperature or ambient temperature is the range from 15° C. to 60° C., such as between 20° C. and 50° C. or 20° C. and 40° C., preferably between 20° C. and 30° C., such as 20° C. and 25° C. In a particular embodiment the room temperature is intended to mean a temperature about 20° C.

The term “Vacuum” means a pressure below 0.02 MPa.

The term “C1-C6 alcohol” means a linear or branched alcohol comprising 1 to 6 carbon atoms. Prefered alcohols are methanol, ethanol, 1-propanol, and 2-propanol.

### DETAILED DESCRIPTION OF THE INVENTION

The present invention is described in more detail in the embodiments below.

E(1) A process for the preparation of 2,2-dimethylpiperazine, characterised in that the process comprises the following steps:
a) Isobutyraldehyde is reacted with a chlorinating agent to form 2-chloro-2-methylpropanal
b) 2-chloro-2-methylpropanal obtained in step a) above is reacted with ethylenediamine in an organic solvent at a temperature between room temperature and reflux temperature of the formed solution to form 6,6-dimethyl-1,2,3,6-tetrahydropyrazine
c) 6,6-dimethyl-1,2,3,6-tetrahydropyrazine obtained in step b) is diluted with C₁-C₆ alcohol and subjected to catalytic hydrogenation to form 2,2-dimethylpiperazine.

E(2) The process according to E(1), wherein step a), before the obtained 2-chloro-2-methylpropanal is reacted with ethylenediamine, is followed by dilution with an organic solvent, addition of catalytic amount of acidic catalyst and heating of the solution to above 90° C.

E(3) The process according to E(1) and E(2), wherein the organic solvent is independently selected from the group consisting of tetrahydrofuran, 2-methyltetrahydrofuran and toluene, or a mixture of said organic solvents.

E(4) The process according to step b) of E(1), wherein the organic solvent is tetrahydrofuran and the temperature is between 55° C. and 66° C.

E(5) The process according to E(2), wherein the organic solvent is toluene.

E(6) The process according to E(1), wherein the C₁-C₆ alcohol is selected from the group consisting of methanol, ethanol, 1-propanol and 2-propanol, and a mixture of two or more of said alcohols.

E(7) The process according to step a) of E(1), wherein the chlorinating agent is selected from the group consisting of chlorine (gas), sulfurylchloride, trichloroisocyanuric acid (TCCA), 1,3-dichloro-5,5-dimethylhydantoin (DCDMI) and N-Chlorosuccinimide (NCS).

E(8) The process according to E(2), wherein the acidic catalyst is selected from the group consisting of sulphuric acid, methanesulfonic acid, p-toluenesulfonic acid, and Montomorillonite K10 (CAS Number: 1318-93-0).

E(9) The process according step a) of E(1), wherein water is added to quench the chlorinating agent and the water subsequently is removed before proceeding with E(2) or step b) of E(1).

E(10) The process according to step b) of E(1), wherein the organic layer containing 6,6-dimethyl-1,2,3,6-tetrahydropyrazine and water are allowed to separate and the lower aqueous layer is discharged.

E(11) The separation according to E(10), wherein said separation takes place at room temperature.

E(12) The process according to step c) of E(1), wherein catalytic hydrogenation takes place in the presence of a Pd/C catalyst.

E(13) A process for the preparation of 2,2-dimethylpiperazine, characterised in that the process comprises the following steps:
a) 2,4,6-tris(2-chloropropan-2-yl)-1,3,5-trioxane is heated to a temperature of above 90° C. in an organic solvent in the presence of an acidic catalyst to obtain 2-chloro-2-methylpropanal
b) 2-chloro-2-methylpropanal obtained in step a) above is reacted with ethylenediamine in an organic solvent at a temperature between room temperature and reflux temperature of the formed solution to form 6,6-dimethyl-1,2,3,6-tetrahydropyrazine
c) 6,6-dimethyl-1,2,3,6-tetrahydropyrazine obtained in step b) is diluted with C₁-C₆ alcohol and subjected to catalytic hydrogenation to form 2,2-dimethylpiperazine.

E(14) The process according to E(13), wherein the organic solvent is independently selected from the group consisting of tetrahydrofuran, 2-methyltetrahydrofuran and toluene, and a mixture of said organic solvents.

E(15) The process according to step b) of E(13), wherein the organic solvent is tetrahydrofuran and the temperature is between 55° C. and 66° C.

E(16) The process according to step a) E(13), wherein the organic solvent is toluene.

E(17) The process according to E(13), wherein the alcohol is selected from the group consisting of methanol, ethanol, 1-propanol, 2-propanol, and a mixture of two or more of said alcohols.

E(18) The process according to step a) of E(13), wherein the acidic catalyst is selected from the group consisting of sulphuric acid, methanesulfonic acid, p-toluenesulfonic acid, and Montomorillonite K10 (CAS Number: 1318-93-0)

E(19) The process according to step b) of E(13), wherein the organic layer containing 6,6-dimethyl-1,2,3,6-tetrahydropyrazine is allowed to separate and the lower aqueos layer is discharged.

E(20) The separation according to E(19), wherein said separation takes place at room temperature at room temperature.

E(21) The process according to step c) of E(13), wherein catalytic hydrogenation takes place in the presence of a Pd/C catalyst.

E(22)The process according to any of claims E(1), E(12), E(13) and E(21), wherein the mixture comprising 6,6-dimethyl-1,2,3,6-tetrahydropyrazine is hydrogenated at a temperature from 40° C. to 80° C. and at a pressure from 0.2MPa to 0.8MPa.

E(23) The process according to any of E(1) and E(13), wherein the formed 2,2-dimethylpiperazine is distilled.

E(24) The process according to any of E(1), E(13) and E(23), wherein 2,2-dimethylpiperazine subsequently is mixed with a suitable acid to provide a 2,2-dimethylpiperazine salt.

E(25) The salt formation according to E(24), wherein the 2,2-dimethylpiperazine salt is selected from the group consisting of tartrate, fumarate, succinate, hydrochloride, oxalate, hydrobromide, hydroiodide, sulfate, p-toluensulfate and maleate.

E(26) The process according to any of E(1) and E(13), wherein the formed 2,2-dimethylpiperazine is reacted with di-tert-butyl dicarbonate in alcohol containing tartaric acid to obtain tert-butyl-3,3-dimethylpiperazine-1-carboxylate hemi-DL-tartrate.

E(27) The salt formation according E(26), wherein the alcohol is selected from the group consisting of methanol, ethanol, 1-propanol, 2-propanol or a mixture of two or more of said alcohols.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

The description herein of any aspect of the invention using terms such as “comprising”, “having,” “including,” or “containing” with reference to an element or elements is intended to provide support for a similar aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the various aspects, embodiments, implementations and features of the invention mentioned herein may be claimed separately, or in any combination.

Experimental Section

The invention is illustrated by the Examples described below. The examples are not intended to limit the scope of the invention. Various modifications and embodiments can be made without departing from the scope and spirit of the invention, which is defined by the following claims only.

Abbreviations
“rt” is room temperature.
“approx.” is approximately
“min” is minutes
“h” is hours
“g” is gram
“L” is liter
“mL” is milliliter
“w/w” is weight per weight.
“v/v” is volume per volume
“GC” is gas chromatography
“ETAM” is ethylendiamine

If not otherwise stated, the purity of the intermediates and product was assessed by GC analysis and the values are expressed in area %.

Gas Chromatography (GC) methods

GC method 1 (analysis of 2,2-dimethyl piperazine and intermediates)

Column: Restek Rtx-5 Amine (length=30 m; Int. Diam.: 0.53 mm; ft.: 3.0 micrometer) or equivalent

Column temperature: 50° C. in isotherm for 5 minutes

Temp. gradient at 20° C./min until 300° C. isotherm at 300° C. for 8 minutes

Run time: 25.5 min

Injection temperature: 250° C. split mode

Split ratio: 5:1

Gas saver: Off

Gas carrier: helium (5.8 mL/min)

Detector: FID

Temp. of detector: 300° C.

Detector Gas: Air (400 mL/min)

H2 (40 mL/min)

Make-up: N2 (25 mL/min)

Injection volume: 1 micro liter

GC method 2 (analysis of tert-butyl-3,3-dimethylpiperazine-1-carboxylate and its hemi-DL-tartrate salt)

Column: Restek Rtx-5 Amine (length=30 m; Int. Diam.: 0.53 mm; ft.: 3.0 micrometer) or equivalent

Column temperature: initial temperature of 50° C.

Temp. gradient of 10° C./min until 140 ° C.; isotherm at 140° C. for 2 min

Temp. gradient of 10° C./min until 240° C. isotherm at 240° C. for 2 min

Temp. gradient of 20° C./min until 300° C. isotherm at 300° C. for 2 minutes Run time: 28 min

Injection temperature: 200° C. split mode

Split ratio: 5:1

Gas carrier: helium (8.0 mL/min)

Detector: FID

Temp. of detector: 300 ° C.

Detector Gas: Air (400 mL/min)

H2 (40 mL/min)

Make-up: N2 (25 mL/min)

Injection volume: 1 micro liter

Nuclear Magnetic Resonance (NMR)

1H-NMR spectrum was recorded at 20° C. on a Bruker Avance 300 or 400. Chemical shifts were reported relative to residual deuterated solvent peaks. The following abbreviations are used for NMR data: s, singlet; bs, broad singlet; d, doublet; t, triplet; m, multiplet.

### EXAMPLE 1

### Preparation of 2,2-dimethylpiperazine with isobutyraldehyde as Starting Material

(Step 1) A reactor was charged at room temperature with sulfuryl chloride (900 kg).

Isobutyraldehyde (471 kg) was added over several hours maintaining the temperature at 20° C.-25° C. To the reaction mixture water (48 L) was added over several hours maintaining the temperature at 30° C.-40° C. followed by the addition of toluene (483L). The purity of the reaction mixture was 71% due to the presence of trimeric (2,4,6-tris(2-chloropropan2-yl)-1,3,5-trioxane) and polymeric forms of 2-chloro-2-methylpropanal. The reaction mixture was heated to 50° C. -55° C. and kept at this temperature for about 1 hour. The layers were allowed to separate and the water layer was discharged. Sulfuric acid (12L) was charged and the reaction mixture heated to reflux for about 6 hours. The purity of the reaction mixture was now 90%. The reaction mixture was cooled to 50-55° C., water (48 L) was added and the reaction mixture was kept at 50° C.-55° C. for about 1 hour. The layers were allowed to separate and the water layer was discharged. The toluene layer containing 2-chloro-2-methylpropanal was cooled to room temperature.

(Step 2) A second reactor was charged with ethylendiamine (1178 kg) and tetrahydrofuran (1887L) and the temperature was adjusted to 60° C. -65° C. To this mixture, 2-chloro-2-methylpropanal in toluene obtained in Step 1 was added over several hours maintaining the temperature at 60° C. -65° C. The reaction mixture was kept at 60° C. -65° C. for another 90 minutes and then cooled to 20° C. -25° C. The layers were allowed to separate and the lower layer was discharged. The organic layer contained the imine intermediate 6,6-dimethyl-1,2,3,6-tetrahydropyrazine.

(Step 3) The organic layer of step 2 containing 6,6-dimethyl-1,2,3,6-tetrahydropyrazine was transferred into a pressure reactor. The transfer lines were rinsed with methanol (481L) and combined with the organic layer. The reactor was charged with 10% Pd/C (42 kg; 50% w/w water). The mixture was hydrogenated at 40° C.-50° C. and 3.5-4 bar until the hydrogen consumption ceased. The temperature was increased to 60-65° C. and hydrogenation was continued for about 2 hours. The conversion of imine into the amine 2,2-dimethylpiperazine was checked by an in-process analysis and the amount of residual imine was found to be 3.5%. Hydrogenation was continued for 7 hours and a second in-process control analysis found 0.9% of residual imine to be present. The reaction mixture was cooled to 20° C.-30° C. and hydrogen and catalyst was removed. The transfer lines were rinsed with methanol (95 L) and combined with the organic layer. The mixture was concentrated by distillation under atmospheric pressure raising the internal temperature gradually up to 120° C.-125° C. The temperature was lowered to 70° C.-80° C. To the residue toluene (471 L) was added. The mixture was concentrated by distillation under atmospheric pressure raising the internal temperature gradually up to 115° C.-125° C. The toluene addition followed by distillation was repeated twice. The residue was cooled to 40-50° C. providing crude 2,2-dimethylpiperazine (purity: 80%). To avoid crystallisation of solid product, methanol (100 L) was added and the solution was further cooled to room temperature.

(Step 4) The solution of crude 2,2-dimethylpiperazine (683 kg) obtained in step 3 was concentrated first under atmospheric pressure then under reduced pressure (0.015 MPa) to remove solvents (toluene, methanol) and the product was distilled under reduced pressure (0.0035-0.0045 MPa). The main fractions (b.p. 64° C.-68° C./0.0035-0.0045 MPa) were united and provided purified 2,2-dimethylpiperazine (165 Kg, purity 95%; the material contained estimated 147 kg of pure product, corresponding to an overall molar yield from isobutyraldehyde of 20%).

### EXAMPLE 2

### Distillation of Crude 2,2-dimethylpiperazine

Crude 2,2-dimethylpiperazine (125 g, containing estimated 69 g of pure product) prepared as described in example 1 was distilled under reduced pressure (0.001-0.0015 MPa). The main fractions (b.p. 35° C.-43° C./0.001-0.0015 MPa) provided purified 2,2-dimethylpiperazine (44 g, purity 98%).

### EXAMPLE 3

### Preparation of 2-chloro-2-methylpropanal (Acid Treatment with Methanesulfonic Acid)

(Step 1) A reactor was charged with sulfuryl chloride (190.9 g) and cooled to 18° C. Isobutyraldehyde (100 g) was added over 2 hours maintaining the temperature at 18° C.-28° C. The reaction mixture was warmed to 30° C.-35° C. and water (10 mL) was added followed by the addition of toluene (103 mL). The mixture was heated to 50° C.-55° C., the layers were allowed to separate and the water layer was discharged. The toluene solution (230.9 g) contained 2-chloro-2-methylpropanal (purity 39%), together with trimeric (2,4,6-tris(2-chloropropan2-yl)-1,3,5-trioxane) and polymeric forms of 2-chloro-2-methylpropanal.

A portion of the solution (57.7 g) was mixed with methanesulfonic acid (0.66 g) and heated to reflux for 6 hours. The reaction mixture was cooled to 50-55° C., water (2.5 mL) was added and the reaction mixture was kept at 50° C.-55° C. for about 30 minutes. The layers were allowed to separate and the water layer was discharged. The toluene layer containing 2-chloro-2-methylpropanal was cooled to room temperature and analysed. The purity of 2-chloro-2-methylpropanal was 87%.

### EXAMPLE 4

### Preparation of 2-chloro-2-methylpropanal with isobutyraldehyde as Starting Material and Comparing Different Chlorinating Agents

2-chloro-2-methylpropanal was produced from isobutyraldehyde with the use of different chlorinating agents. The following chlorinating agents were applied: sulfuryl chloride, trichloroisocyanuric acid (TCCA), 1,3-dichloro-5,5-dimethylhydantoin (DCDMI) and N-Chlorosuccinimide (NCS). The purity of the obtained 2-chloro-2-methylpropanol is listed in table 1:

TABLE 1
Purity (area % GC) of 2-chloro-2-methylpropanol

| | Purity of 2-chloro-2-methylpropanol |
|---|---|
| Chlorinating agent | (area % GC) |
| Sulfuryl chloride | 39-82% (without acid treatment for |
| | depolymerisation) |
| TCCA | 10% |
| DCDMI | 35% |
| NCS | 45% |

The results show that sulfuryl chloride gives the highest degree of purity when converting isobutyraldehyde to 2-chloro-2-methylpropanol, but also that other chlorinating agents may be used.

### EXAMPLE 5

### Preparation of Solid 2-chloro-2-methylpropanal trimer (2,4,6-tris(2-chloropropan2-yl)-1,3,5-trioxane)

A reactor was charged with sulfuryl chloride (191 g) and cooled to 18° C. Isobutyraldehyde (100 g) was added over 2.7 hours maintaining the temperature at 18-28° C. After 30 minutes water (10 mL) was slowly added. The mixture was stirred at 20° C.-25° C. for 20 minutes. The layers were allowed to separate, the water layer was discharged obtaining 2-chloro-2-methylpropanal (156 g).

A portion (50 g) was cooled to 0° C.-5° C. and sulfuric acid (1 mL) was added. The mixture, a dense suspension, was warmed to room temperature and 2-propanol (100 mL) was added. The mixture was heated to 55° C.-60° C. and water (50 mL) was slowly added. The white suspension was cooled to room temperature and left stirring overnight. The suspension was filtered, washed with water (2 times 50 mL) and dried under vacuum at 45° C. providing 33.9 g of 2,4,6-tris(2-chloropropan2-yl)-1,3,5-trioxane (purity 99%, molar yield of 72% from isobutyraldehyde).

### EXAMPLE 6

Preparation of 2-chloro-2-methylpropanal from trimer 2,4,6-tris(2-chloropropan2-yl)-1,3,5-trioxane (Depolymerisation)

Solid trimer 2,4,6-tris(2-chloropropan2-yl)-1,3,5-trioxane obtained as described in example 5 was depolymerised in the presence of an acid catalyst. and toluene to provide 2-chloro-2-methylpropanal. Various acid catalysts (sulfuric acid, methanesulfonic acid, p-toluenesulfonic acid, Montmorillonite K10 (CAS Number: 1318-93-0)) were tested with satisfactory outcome with a typical purity of 90%-91% of 2-chloro-2-methylpropanal. 2-Chloro-2-methylpropanal was transformed into 2,2-dimethylpiperazine by reductive amination as described in Example 1.

### EXAMPLE 7

### Preparation of 2,2-dimethylpiperazine-DL-tartrate from purified 2,2-dimethylpiperazine

A solution of distilled 2,2-dimethylpiperazine (10 g, 0.088 mol) in toluene (6.9 g) was mixed with 2-propanol (100 mL) and heated to about 55-56° C. Solid DL-tartaric acid (6.6 g; 0.044 mol) was added in portions mantaining the temperature at 54-57° C. The mixture was allowed to cool to room temperature and the suspension was stirred overnight. The product was isolated by filtration, washed with 2-propanol (20 mL) and dried under vacuum at 50° C. Pure 2,2-dimethylpiperazine -DL-tartrate (1:1 salt) (10.5 g, 45% molar yield) was obtained as a white solid. 1H-NMR (DMSO-d6 with a drop of D₂O): 6(ppm) 1.21 (6H, s), 2.63 (2H, s), 2.76-2.82 (2H, m), 2.88-2.94 (2H, m), 3.91 (1H, s, tartrate).

Tartaric acid was dosed with the aim of producing the hemi-tartrate salt of 2,2-dimethylpiperazine, but surprisingly only the mono tartrate was formed.

### Example 8

### Preparation of 2,2-dimethylpiperazine-DL-tartrate from crude 2,2-dimethylpiperazine

A solution (17.7 g) of crude 2,2-dimethylpiperazine (containing estimated 10 g, 0.088 mol) in toluene was mixed with 2-propanol (100 mL) and heated to about 55-56° C. Solid DL-tartaric acid (6.6 g; 0.044 mol) was added. The mixture was allowed to cool to room temperature and the suspension was stirred overnight. The product was isolated by filtration, washed with 2-propanol (20 mL) and dried under vacuum at 50° C. Pure 2,2-dimethylpiperazine DL-tartrate (9.9 g, 43% molar yield) was obtained as a white solid. The 1H-NMR was consistent with the one of experiment 7.

Tartaric acid was dosed with the aim of producing the hemi-tartrate salt of 2,2-dimethylpiperazine, but surprisingly only the mono tartrate was formed.

### EXAMPLE 9

### Preparation of 2,2-dimethylpiperazine DL-tartrate from purified 2,2-dimethylpiperazine

Distilled 2,2-dimethylpiperazine (10 g, 0.088 mol) was mixed with 2-propanol (100 mL) and heated to about 56° C.-57° C. Solid DL-tartaric acid (13.1 g; 0.087 mol) was added. The mixture was kept at 56° C.-57° C. for 1 hour and then allowed to cool to room temperature. The suspension was stirred overnight. The product was isolated by filtration, washed with 2-propanol (20 mL) and dried under vacuum at 50° C. Pure 2,2-dimethylpiperazine DL-tartrate (18.5 g, 80% molar yield) was obtained as a white solid. The 1H-NMR was consistent with the one of experiment 7.

### EXAMPLE 10

### Preparation of 2,2-dimethylpiperazine Salts

In a manner similar to the preparation of DL-tartrate as described in Examples 7-9 three other salts: succinate, oxalate and fumarate were prepared (all 1:1 salts). 1H NMR data are provided below:

2,2-Dimethylpiperazine succinate (1:1 salt), 1H-NMR (DMSO-d6): 6(ppm) 1.20 (6H, s), 2.27 (4H, s, succinate), 2.61 (2H, s), 2.73-2.79 (2H, m), 2.85-2.91 (2H, m).

2,2-Dimethylpiperazine oxalate (1:1 salt), 1H-NMR (D₂O): 6(ppm) 1.48 (6H, s), 3.32 (2H, s), 3.39-3.45 (2H, m), 3.49-3.54 (2H, m).

2,2-Dimethylpiperazine fumarate (1:1 salt), 1H-NMR (D₂O): 6(ppm) 1.49 (6H, s), 3.35 (2H, s), 3.41-3.47 (2H, m), 3.51-3.57 (2H, m), 6.55 (2H, s, fumarate).

### EXAMPLE 11

### Preparation of tert-butyl-3,3-dimethylpiperazine-1-carboxylate hemi-DL-tartrate

2,2-dimethylpiperazine (20 g containing 17.8 g pure material, 0.156 mol) was dissolved in denatured ethanol (40 mL) and the solution was heated to approximately 50° C. Di-tert-butyl dicarbonate (38.2 g, 0.175 mol) was dissolved in denatured ethanol (68 mL). The solution of di-tertbutyl dicarbonate was added to the solution of 2,2-dimethylpiperazine over a period of 1 hour. Denatured ethanol (12 mL) was added and the solution was cooled to 20° C. The solution was transferred to a suspension of DL-tartaric acid (13.1 g, 0.0876 mol) in denatured ethanol (80 mL) and warmed to 53° C. Deanatured ethanol (10 mL) was added and the reaction mixture was heat to reflux for 30 minutes. The mixture was cooled slowely to 12° C. The precipitate was filtered off and washed with denatured ethanol (60 mL). The solid was dried in a vacuum oven at 50° C. to yield tert-butyl-3,3-dimethylpiperazine-1-carboxylate hemi-DL-tartrate (39.7 g, molar yield 88%) with 99.9% purity according to GC analysis.

### Example 12

Preparation of tert-butyl-3,3-dimethylpiperazine-1-carboxylate hemi-DL-tartrate

In a reactor 2,2-dimethylpiperazine (91 kg containing 84.2 kg pure material, 737 mol) was dissolved in denatured ethanol (354 L) and the solution was heated to approximately 50° C. Separately di-tert-butyl dicarbonate (180 kg, 826 mol) was dissolved in denatured ethanol (190 L). The solution of di-tertbutyl dicarbonate was added to the solution of 2,2-dimethylpiperazine over a period of 3 hours. Denatured ethanol (63 L) was added and the solution was kept at 50° C. for further 2 hours. The solution, containing tert-butyl-3,3-dimethylpiperazine-1-carboxylate was then cooled to 20° C. A second reactor was charged with DL-tartaric acid (60 kg, 400 mol) denatured ethanol (633 L) and water (18 L). The mixture was heated to reflux to achieve a solution. The temperature was lowered to 72° C.-75° C., and the solution was blank filtered through a filter cartridge. Solvent (240 kg) was distilled off under vacuum maintaing the temperature below 45° C. The temperature was then lowered to 20° C.-30° C. obtaining a suspension of tartaric acid in denatured ethanol. The solution, containing tert-butyl-3,3-dimethylpiperazine-1-carboxylate was blank filtered and combined with the suspension of tartaric acid in denatured ethanol. Denatured ethanol (50 L) was added and the mixture was heated to about 70° C. After 30 minutes the suspension was cooled slowly to 12° C. The precipitate was filtered off and washed with denatured ethanol (3 times 95L). The solid was dried in under vacuum at 50° C. to yield tert-butyl-3,3-dimethylpiperazine-1-carboxylate hemi-DL-tartrate (187 kg, molar yield 88%) with 100% purity according to GC analysis.

1. A process for the preparation of 2,2-dimethylpiperazine, characterised in that the process comprises the following steps:
a) Isobutyraldehyde is reacted with a chlorinating agent to form 2-chloro-2-methylpropanal
b) 2-chloro-2-methylpropanal obtained in step a) above is reacted with ethylenediamine in an organic solvent at a temperature between room temperature and reflux temperature to form 6,6-dimethyl-1,2,3,6-tetrahydropyrazine
c) 6,6-dimethyl-1,2,3,6-tetrahydropyrazine obtained in step b) is diluted with C₁-C₆ alcohol and subjected to catalytic hydrogenation to form 2,2-dimethylpiperazine.

2. The process according to claim 1, wherein step a), before the obtained 2-chloro-2-methylpropanal is reacted with ethylenediamine, is followed by dilution with an organic solvent, addition of catalytic amount of acidic catalyst and heating of the solution to above 90° C. to transform formed trimeric and polymeric forms of 2-chloro-2-methylpropanal into monomeric 2-chloro-2-methylpropanal before the obtained 2-chloro-2-methylpropanal is reacted with ethylenediamine.

3. The process according to claim 1, wherein the organic solvent is independently selected from the group consisting of tetrahydrofuran, 2-methyltetrahydrofuran and toluene, and a mixture of said organic solvents.

4. The process according to claim 1, wherein the C₁-C₆ alcohol is selected from methanol, ethanol, 1-propanol and 2-propanol, and a mixture of two or more of said alcohols.

5. The process according to step a) of claim 1, wherein the chlorinating agent is selected from the group consisting of chlorine (gas), sulfurylchloride, trichloroisocyanuric acid (TCCA), 1,3-dichloro-5,5-dimethylhydantoin (DCDMI) and N-Chlorosuccinimide (NCS).

6. The process according to claim 2, wherein the acidic catalyst is selected from the group consisting of sulphuric acid, methanesulfonic acid, p-toluenesulfonic acid, and Montomorillonite K10 (CAS Number: 1318-93-0).

7. The process according step a) of claim 1, wherein water is added to quench the chlorinating agent and the water subsequently is removed before proceeding with step b) of claim 1.

8. The process according to step b) of claim 1, wherein the organic layer containing 6,6-dimethyl-1,2,3,6-tetrahydropyrazine and water are allowed to separate and the lower aqueous layer is discharged.

9. The process according to step c) of claim 1, wherein catalytic hydrogenation takes place in the presence of a Pd/C catalyst.

10. A process for the preparation of 2,2-dimethylpiperazine, characterised in that the process comprises the following steps:
a) 2,4,6-tris(2-chloropropan-2-yl)-1,3,5-trioxane is heated to a temperature of above 90° C. in an organic solvent in the presence of an acidic catalyst to obtain 2-chloro-2-methylpropanal
b) 2-chloro-2-methylpropanal obtained in step a) above is reacted with ethylenediamine in an organic solvent at a temperature between room temperature and reflux temperature to form 6,6-dimethyl-1,2,3,6-tetrahydropyrazine
c) 6,6-dimethyl-1,2,3,6-tetrahydropyrazine obtained in step b) is diluted with C₁-C₆ alcohol and subjected to catalytic hydrogenation to form 2,2-dimethylpiperazine.

11. The process according to claim 10, wherein the organic solvent is independently selected from the group consisting of tetrahydrofuran, 2-methyltetrahydrofuran and toluene, and a mixture of said organic solvents.

12. The process according to claim 10, wherein the alcohol is selected from the group consisting of methanol, ethanol, 1-propanol, 2-propanol, and a mixture of two or more of said alcohols.

13. The process according to step a) of claim 10, wherein the acidic catalyst is selected from the group consisting of sulphuric acid, methanesulfonic acid, p-toluenesulfonic acid, and Montomorillonite K10 (CAS Number: 1318-93-0)

14. The process according to step b) of claim 10, wherein the organic layer containing 6,6-dimethyl-1,2,3,6-tetrahydropyrazine is allowed to separate at room temperature and the lower aqueous layer is discharged.

15. The process according to step c) of claim 10, wherein catalytic hydrogenation takes place in the presence of a Pd/C catalyst.

16. The process according to claim 1, wherein the mixture comprising 6,6-dimethyl-1,2,3,6-tetrahydropyrazine is hydrogenated at a temperature from 40° C. to 80° C. and at a pressure from 0.2 MPa to 0.8 MPa.

17. The process according to claim 1, wherein the formed 2,2-dimethylpiperazine is distilled.

18. The process according to claim 1, wherein 2,2-dimethylpiperazine is mixed with a suitable acid to provide a 2,2-dimethylpiperazine salt.

19. The process according to claim 18, wherein the 2,2-dimethylpiperazine salt is selected from the group consisting of tartrate, fumarate, succinate, hydrochloride, oxalate, hydrobromide, hydroiodide, sulfate, p-toluensulfate and maleate.

20. The process according to claim 1, wherein the formed 2,2-dimethylpiperazine is reacted with di-tert-butyl dicarbonate in alcohol containing tartaric acid to obtain tert-butyl-3,3-dimethylpiperazine-1-carboxylate hemi-DL-tartrate.

21. The process according to claim 20, wherein the alcohol is selected from the group consisting of methanol, ethanol, 1-propanol, 2-propanol and a mixture of two or more of said alcohols.
